(19) United States
(12) Patent Application Publication
(10) Pub. No.: US 2020/0268644 A1
(43) Pub. Date: Aug. 27, 2020

(54) METHOD FOR PREPARING LOW-MOLECULAR WEIGHT HORSE PLACENTA ENZYME HYDROLYSATE WITH ANTI-WRINKLING ACTIVITY

(71) Applicant: SJ INTERNATIONAL CO., LTD, Chuncheon-si, Gangwon-do (KR)

(72) Inventors: Yong Hwan OH, Chuncheon-si, Gangwon-do (KR); Myeon CHOE, Chuncheon-si, Gangwon-do (KR); Kyoung Kon KIM, Chuncheon-si, Gangwon-do (KR); Tae Woo KIM, Gangwon-do (KR)

(73) Assignee: SJ INTERNATIONAL CO., LTD, Chuncheon-si, Gangwon-do (KR)

(21) Appl. No.: 15/781,484

(22) PCT Filed: Feb. 19, 2018
(86) PCT No.: PCT/KR2018/001989

§ 371 Date: Jun. 5, 2018

(30) Foreign Application Priority Data
Feb. 17, 2017 (KR) 10-2017-0021650

(51) Int. Cl. A61K 8/98; A61Q 19/08

(52) U.S. Cl. CPC A61K 8/982; A61Q 19/08

(57) ABSTRACT

Disclosed is a method for preparing a low-molecular weight horse placenta enzyme hydrolysate. According to the present invention, through development of pretreatment and optimal enzymatic treatment, the horse placenta extract can be modified to a low-molecular weight. The low-molecular weight horse placenta hydrolysate obtained according to the present invention can exhibit excellent skin permeability due to low molecular weight.

### TECHNICAL FIELD

The present invention relates to a method for preparing a low-molecular weight horse placenta enzyme hydrolysate and more specifically, to a method for preparing a low-molecular weight horse placenta enzyme hydrolysate with anti-wrinkling activity.

### BACKGROUND ART

The skin is the outer cover of the body consisting of the epidermis, dermis and subcutaneous fat, functions as a physical barrier to protect the body against various environmental factors and as a tactile sensor, and controls body temperature. However, in industrialized modern societies, westernized lifestyles, stress and an increase in UV resulting from environmental pollution cause skin damage and aging.

Meanwhile, skin aging is divided into endogenous aging which results from reduction in hormone secretion over time, deterioration of functions and activities of immune cells and thus degradation of biosynthesis of proteins constituting the human body, and exogenous aging resulting from contaminated air, drugs, UV and the like (Park H R et al., Journal of Investigative Cosmetology. 7: 115-122. 2011).

Among factors of exogenous aging, ultraviolet light causes loss in flexibility of the stratum corneum on the surface of the skin, and makes the skin dry and rough. In addition, ultraviolet light inhibits synthesis of collagen and improves the expression of extracellular matrix proteases, matrix metalloproteinases (MMPs) (Jin Young Seo et al., Korean Journal of Investigative Dermatology. 8(4):187-194. 2001). For this reason, as the amount of collagen in the skin is lacking and elastic fibers are modified in nature, wrinkles are generated.

Retinol is the most commonly used substance to alleviate skin wrinkles. Retinol is a wrinkle-functioning drug ingredient announced by the Ministry of Food and Drug Safety, which is known to regulate the differentiation and regeneration of epidermal cells (Griffiths C E et al., The New England Journal of Medicine 329 (8): 530-535). However, despite its excellent effect, retinol has low heat stability, is easily deformed when heat is applied, and has high toxicity, which causes skin irritation.

In order to solve the instability of retinol, various natural substances such as paeoniflorin extracted from peony as an ingredient for controlling the extracellular matrix (ECM) and compound K present in ginseng are used as materials for alleviating skin wrinkles.

However, peony and ginseng, which are the raw materials of these substances, are disadvantageously expensive and thus have poor price competition. There is a need to develop physiologically active substances having high price competitiveness and excellent efficacy in that continuous supply and acquisition of raw materials are required for food and cosmetic compositions for improving skin wrinkles.

Recently, the horse placenta extracts have found to have anti-pigmentation activity and whitening activity (Mallick Set el., Pigment Cell Res. February 18 (1), 25-33, 2005), as well as inhibition and reduction of wrinkles. Accordingly, these extracts are used as raw materials for cosmetics and food, but the horse placenta extract has disadvantages of being easily degraded due to peculiar odor and instability, and causing skin irritation when used at a high concentration. In addition, the horse placenta extract has a drawback of poor skin permeability because it is made of a polymer material.

### DISCLOSURE

### Technical Problem

Therefore, the present invention has been made in view of the above problems, and it is an object of the present invention to provide a method for modifying a horse placenta extract into a low-molecular weight in an attempt to solve the conventional problems of horse placenta extracts such as low skin permeability due to polymer ingredient present therein.

### Technical Solution

In accordance with the present invention, the above and other objects can be accomplished by the provision of a method for preparing a horse placenta hydrolysate including simultaneously treating a horse placenta with a commercially available enzyme called “Alcalase 2.4 L” and a commercially available enzyme called “Bromelain BR 1200”.

Regarding the method for preparing a horse placenta hydrolysate, the horse placenta is preferably treated with at least one of Alcalase 2.4 L or Bromelain BR 1200 for 12 to 24 hours.

Regarding the method for preparing a horse placenta hydrolysate, the horse placenta is preferably further treated with an enzyme called “Flavourzyme”.

Regarding the method for preparing a horse placenta hydrolysate, the horse placenta is preferably treated with Flavourzyme after simultaneously treating with the Alcalase 2.4 L and the Bromelain BR 1200.

Preferably, the method may further include pretreatment of adding an alkali before the enzymatic treatment.

Regarding the method for preparing a horse placenta hydrolysate, preferably, the alkali is KOH.

Regarding the method for preparing a horse placenta hydrolysate, preferably, the horse placenta hydrolysate has a molecular weight of 2,000 to 2,500 Da.

### Advantageous Effects

According to the present invention, the horse placenta hydrolysate can be efficiently modified to a low-molecular weight through pretreatment and optimal enzymatic treatment. The low-molecular weight horse placenta hydrolysate obtained by the present invention can exhibit excellent skin permeability due to low molecular weight. Accordingly, the horse placenta hydrolysate according to the present invention is considerably suitable for use as an ingredient for cosmetics.

### BRIEF DESCRIPTION OF THE DRAWINGS

The above and other objects, features and other advantages of the present invention will be more clearly understood from the following detailed description taken in conjunction with the accompanying drawings, in which:

FIG. 1 shows RSM analysis results of Alcalase;

FIG. 2 shows RSM analysis results of Papain T100MG;

FIG. 3 shows RSM analysis results of FoodPro Alkaline Protease;

FIG. 4 shows RSM analysis results of Bromelain BR 1200;

FIG. 5 shows measurement results of procollagen synthesis activity depending on type of combined enzymes;

FIG. 6 shows measurement results of procollagen synthesis activity depending on treatment time of Alcalase-Bromelain combined enzyme;

FIG. 7 shows measurement results of procollagen synthesis activity involved in treatment of Flavourzyme as an exo-type protease;

FIG. 8 shows measurement results of procollagen synthesis activity after treatment with KOH before enzymatic treatment;

FIG. 9 shows comparison results of molecular weights of positive control (JAPAN) and placenta hydrolysate prepared in Example 4 according to the present invention; and

FIG. 10 shows comparison results of procollagen synthesis activity of positive control (JAPAN) and the placenta hydrolysate prepared in Example 4 according to the present invention.

### BEST MODE

Horse placenta has a drawback of low skin permeability due to high molecular weight. This causes a problem of limiting the use range of horse placenta with superior skin activity as a cosmetic ingredient. Accordingly, in an attempt to check the functionality of horse placenta according to the present invention, the horse placenta is modified to a low molecular weight at high yield by enzymatic treatment or pretreatment, and the procollagen synthesis activity of the hydrolysate is measured.

First, optical extraction conditions of horse placenta hydrolysate were established by response surface methodology (RSM) analysis of four endopeptidases, called “Alcalase 2.4 L”, “Papain T100MG”, “FoodPro Alkaline Protease”, and “Bromelain BR 1200”.

Results of test by RSM showed that Alcalase 2.4 L had an optimal hydrolysis yield of 72.5446% at an enzyme concentration of 0.5% (w/w) for a treatment time of 24 hours at pH 8.0. In addition, Papain T100MG had an optimal hydrolysis yield of 29.6816% at an enzyme concentration of 0.2% (w/w) for treatment time 14.4747 hours at pH 5.0. In addition, FoodPro Alkaline Protease had an optimal hydrolysis yield of 74.5883% at an enzyme concentration of 1.0% (w/w) for a treatment time 21.4444 hours at pH 8.0. In addition, Bromelain BR 1200 had an optimal hydrolysis yield of 26.8868% at an enzyme concentration of 0.05% (w/w) for a treatment time 24 hours at pH 8.0.

Meanwhile, according to the present invention, hydrolysates are obtained by treatment with a combination of the four endopeptidases and then the ability of the hydrolysates to synthesize procollagen in vivo (hereinafter referred to as “procollagen synthesis activity”) was measured. Test results confirmed that hydrolysate samples obtained by treating with both “Alcalase 2.4 L” and “Bromelain BR 1200” exhibited better procollagen synthesis activity than other hydrolysate samples.

Based on these results, the present invention provides a method for preparing a horse placenta hydrolysate according to the present invention including treating the horse placenta simultaneously with a commercially available enzyme called “Alcalase 2.4 L”, and a commercially available enzyme called “Bromelain BR 1200”.

The “horse placenta” herein used is derived from a horse, which is directly obtained from the horse or is commercially available.

Meanwhile, regarding the present invention, at least one of the Alcalase 2.4 L or the Bromelain BR 1200 is preferably treated for 12 to 24 hours. When one is treated for about 12 hours, higher procollagen synthesis activity is obtained, as compared to control group (100%). When treatment is carried out for longer than 24 hours, economically disadvantageously, an increase in efficacy as compared to treatment time is insufficient. More preferably, both the enzymes are treated for 12 to 24 hours because samples treated with the two enzymes for 12 hours exhibit the highest procollagen synthesis activity.

Meanwhile, the method for preparing a horse placenta hydrolysate according to the present invention preferably further includes treatment with an enzyme called “Flavourzyme”. In this case, preferably, the treatment with “Flavourzyme” is conducted after treatment with Alcalase 2.4 L and Bromelain BR 1200. The Flavourzyme is an exo-type protease. When further treating with Flavourzyme, procollagen synthesis activity is increased, as when not treating therewith.

Meanwhile, preferably, the method for preparing a horse placenta hydrolysate according to the present invention preferably includes pretreatment of adding an alkali before treatment with enzymes. The reason for this is that, when pretreatment is carried out by adding an alkali before the enzymatic treatment, higher procollagen synthesis activity is obtained, as compared to when the pretreatment is not carried out. At this time, the alkali is preferably KOH, more preferably at about 0.1 to 1.0M.

Meanwhile, the horse placenta hydrolysate according to the present invention preferably has a molecular weight of 2,000 to 2,500 Da. When the method according to the present invention is used, a hydrolysate having a molecular weight of 2,000 to 2,500 Da can be obtained from the horse placenta. The hydrolysate having the molecular weight within this range exhibits excellent procollagen synthesis activity, as compared to the control group.

Hereinafter, the present invention will be described in more detail with reference to the following Example and Test example, and the scope of the present invention is not limited to the Example and Test example, and includes variations of technical concepts equivalent thereto.

### Example 1: Search for Optimal Treatment Conditions of Four Endopeptidases

### 1. Test Object

Alcalase 2.4 L, Papain T100MG, FoodPro Alkaline Protease and Bromelain BR 1200, which are four domestically sold industrial proteases, were purchased and optimal conditions of the yield to obtain hydrolysates from the horse placenta were researched.

In order to select optimal levels of enzyme concentration, reaction time and reaction pH upon enzymatic treatment of the horse placenta, test conditions were chosen by a Box-Behnken method, using a response surface methodology (RSM) computer program (Minitab 17.1.0, Minitab Inc, USA). Testing was conducted under test conditions and ranges each suitable for enzymes.

### 2. Test Method

### (1) Enzymatic Treatment

\textcircled{1} RSM test plans were established by setting enzyme-dependent independent variables (enzyme concentration, reaction time and pH).
\textcircled{2} Enzymes were added after pH adjustment depending on conditions.
\textcircled{3} Enzymatic treatment was conducted under respective conditions at 80° C. for 20 minutes and then deactivated.
\textcircled{4} The deactivated enzymatic treatment product was centrifuged at 8,000 rpm for 10 minutes and the resulting supernatant was harvested.

### (2) Yield Measurement

\textcircled{1} A substance with a size of 5 kDa or less was separated from the supernatant harvested after enzymatic treatment using a filter Vivaspin Turbo 4 (5 kDa).
\textcircled{2} 3 g of the enzymatic treatment solution was added to Vivaspin Turbo 4 and the resulting mixture was centrifuged at 7,500 RCF for 60 minutes.
\textcircled{3} After centrifugation, the bottom part passing through a sieve of 5 kDa was lyophilized.
\textcircled{4} The weight of the lyophilized bottom part was measured and the difference between the weight after test and the weight before test was then calculated.
\textcircled{5} Yield was calculated in accordance with the following Equation 1:

$$Weight after kDa pass \div ( Weight of solid content of raw material \times \frac{Weight of harvested enzyme treatment product}{Total wegith of enzyme treatment product ( Raw material + D . W} ) \qquad [ Equation 1 ]$$

### 3. Test Results

### (1) Alcalase

As shown in the following Table 1, the enzyme concentration test range was 0.2 to 0.5% (w/w), test time was 1 to 24 hours and pH was 6.5 to 8.

TABLE 1

| | | | | Enzyme | | | |
|---|---|---|---|---|---|---|---|
| StdOrder | RunOrder | PtType | Blocks | concentration | Time | pH | % |
| 4 | 1 | 2 | 1 | 0.5 | 24 | 7.25 | 64.59436 |
| 2 | 2 | 2 | 1 | 0.5 | 1 | 7.25 | 27.44709 |
| 1 | 3 | 2 | 1 | 0.2 | 1 | 7.25 | 21.8254 |
| 10 | 4 | 2 | 1 | 0.35 | 24 | 6.5 | 40.23369 |
| 12 | 5 | 2 | 1 | 0.35 | 24 | 8 | 66.57848 |
| 8 | 6 | 2 | 1 | 0.5 | 12.5 | 8 | 63.60229 |
| 7 | 7 | 2 | 1 | 0.2 | 12.5 | 8 | 61.17725 |
| 11 | 8 | 2 | 1 | 0.35 | 1 | 8 | 53.68166 |
| 9 | 9 | 2 | 1 | 0.35 | 1 | 6.5 | 27.44709 |
| 5 | 10 | 2 | 1 | 0.2 | 12.5 | 6.5 | 38.69048 |
| 15 | 11 | 0 | 1 | 0.35 | 12.5 | 7.25 | 52.68959 |
| 13 | 12 | 0 | 1 | 0.35 | 12.5 | 7.25 | 46.51675 |
| 6 | 13 | 2 | 1 | 0.5 | 12.5 | 6.5 | 46.40653 |
| 3 | 14 | 2 | 1 | 0.2 | 24 | 7.25 | 57.20899 |
| 14 | 15 | 0 | 1 | 0.35 | 12.5 | 7.25 | 47.28836 |

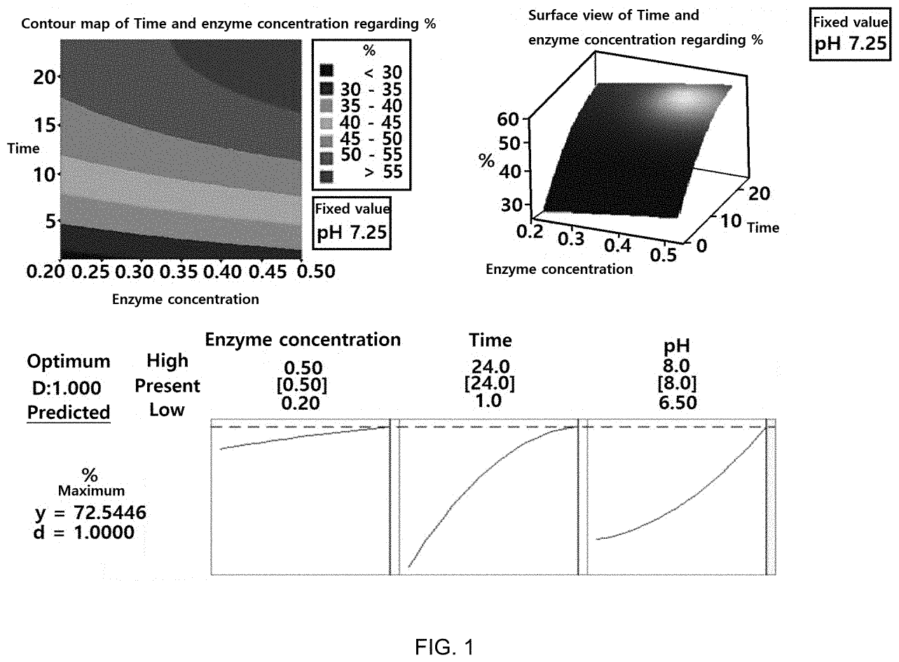

Test results are shown in Table 1 and FIG. 1. It could be seen based on these test results that an optimal yield of 72.5446% could be obtained under the conditions of an enzyme concentration of 0.5% (w/w), a treatment time of 24 hours and pH 8.0.

### (2) Papain T100MG

As shown in the following Table 2, the enzyme concentration test range was 0.01 to 0.2% (w/w), test time was 1 to 24 hours and pH was 5 to 7.5.

TABLE 2

| | | | | Enzyme | | | |
|---|---|---|---|---|---|---|---|
| StdOrder | RunOrder | PtType | Blocks | concentration | Time | pH | % |
| 5 | 1 | 2 | 1 | 0.01 | 12.5 | 5 | 9.589947 |
| 8 | 2 | 2 | 1 | 0.2 | 12.5 | 7.5 | 22.48677 |
| 11 | 3 | 2 | 1 | 0.105 | 1 | 7.5 | 7.495591 |
| 4 | 4 | 2 | 1 | 0.2 | 24 | 6.25 | 20.7231 |
| 13 | 5 | 0 | 1 | 0.105 | 12.5 | 6.25 | 18.29806 |
| 1 | 6 | 2 | 1 | 0.01 | 1 | 6.25 | 3.306878 |
| 10 | 7 | 2 | 1 | 0.105 | 24 | 5 | 19.17989 |
| 3 | 8 | 2 | 1 | 0.01 | 24 | 6.25 | 8.818342 |
| 2 | 9 | 2 | 1 | 0.2 | 1 | 6.25 | 15.10141 |
| 12 | 10 | 2 | 1 | 0.105 | 24 | 7.5 | 16.42416 |
| 9 | 11 | 2 | 1 | 0.105 | 1 | 5 | 13.77866 |
| 14 | 12 | 0 | 1 | 0.105 | 12.5 | 6.25 | 22.92769 |
| 6 | 13 | 2 | 1 | 0.2 | 12.5 | 5 | 30.97443 |
| 7 | 14 | 2 | 1 | 0.01 | 12.5 | 7.5 | 8.597884 |
| 15 | 15 | 0 | 1 | 0.105 | 12.5 | 6.25 | 22.48677 |

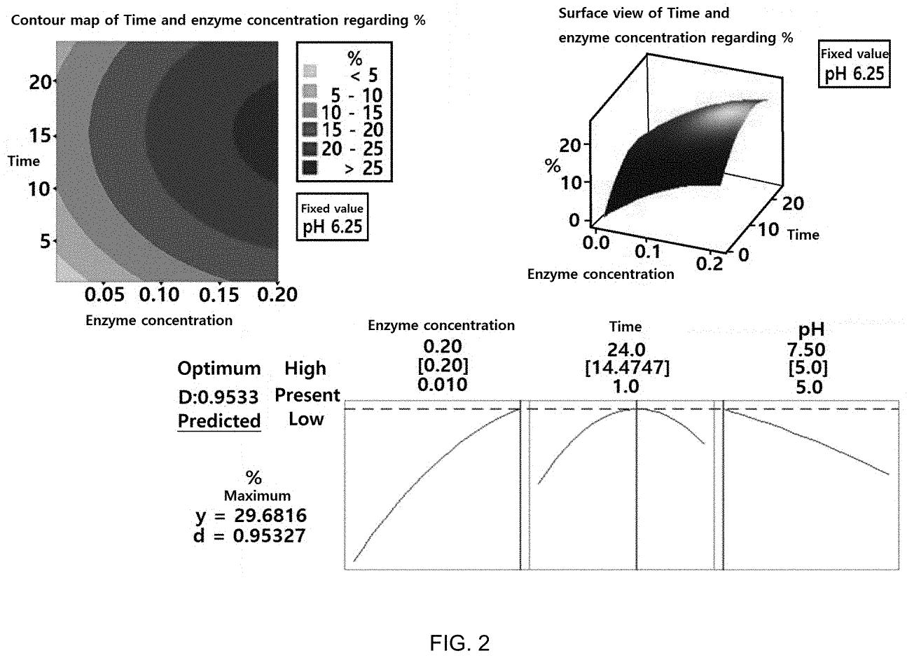

Test results are shown in Table 2 and FIG. 2. It could be seen based on these test results that an optimal yield of 29.6816% could be obtained under the conditions of an enzyme concentration of 0.2% (w/w), a treatment time of 14.4747 hours and pH 5.0.

### (3) FoodPro Alkaline Protease

As shown in the following Table 3, the enzyme concentration test range was 0.25 to 1% (w/w), test time was 1 to 24 hours and pH was 7.5 to 9.

TABLE 3

| | | | | Enzyme | | | |
|---|---|---|---|---|---|---|---|
| StdOrder | RunOrder | PtType | Blocks | concentration | Time | pH | % |
| 12 | 1 | 2 | 1 | 0.625 | 24 | 9 | 54.12257 |
| 8 | 2 | 2 | 1 | 1 | 12.5 | 9 | 63.60229 |
| 2 | 3 | 2 | 1 | 1 | 1 | 8.25 | 25.68342 |
| 11 | 4 | 2 | 1 | 0.625 | 1 | 9 | 29.98236 |
| 1 | 5 | 2 | 1 | 0.25 | 1 | 8.25 | 22.81746 |
| 4 | 6 | 2 | 1 | 1 | 24 | 8.25 | 76.27866 |
| 9 | 7 | 2 | 1 | 0.625 | 1 | 7.5 | 30.09259 |
| 14 | 8 | 0 | 1 | 0.625 | 12.5 | 8.25 | 64.81481 |
| 5 | 9 | 2 | 1 | 0.25 | 12.5 | 7.5 | 53.13051 |
| 13 | 10 | 0 | 1 | 0.625 | 12.5 | 8.25 | 53.4612 |
| 15 | 11 | 0 | 1 | 0.625 | 12.5 | 8.25 | 65.36596 |
| 10 | 12 | 2 | 1 | 0.625 | 24 | 7.5 | 62.72046 |
| 7 | 13 | 2 | 1 | 0.25 | 12.5 | 9 | 43.65079 |
| 3 | 14 | 2 | 1 | 0.25 | 24 | 8.25 | 39.903 |
| 6 | 15 | 2 | 1 | 1 | 12.5 | 7.5 | 61.7284 |

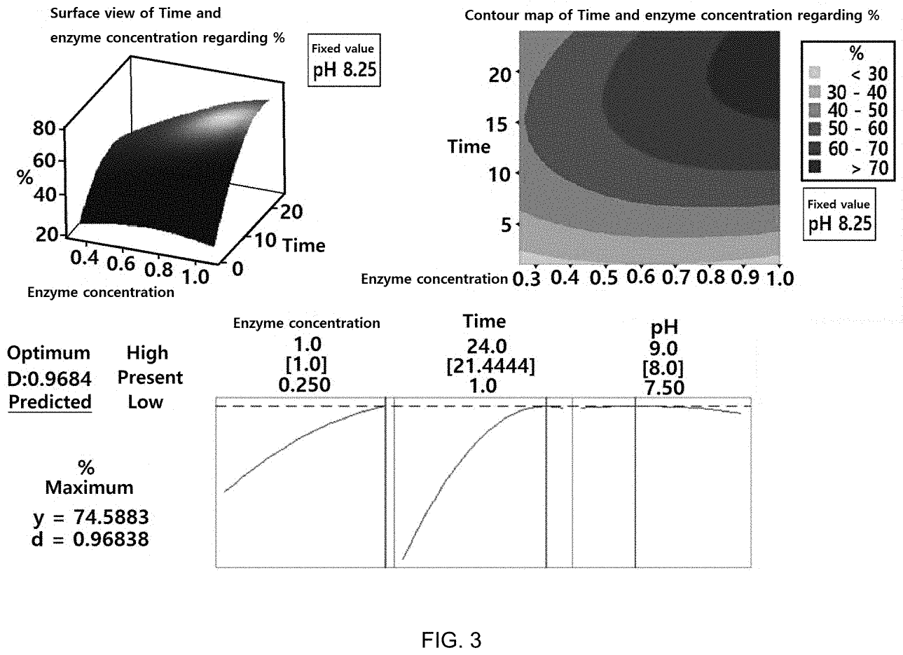

Test results are shown in Table 3 and FIG. 3. It could be seen based on these test results that an optimal yield of 74.5883% could be obtained under the conditions of an enzyme concentration of 1.0% (w/w), a treatment time of 21.4444 hours and pH 8.0.

### (4) Bromelain BR 1200

As shown in the following Table 4, the enzyme concentration test range was 0.005 to 0.05% (w/w), test time was 1 to 24 hours and pH was 6 to 8.

TABLE 4

| | | | | Enzyme | | | |
|---|---|---|---|---|---|---|---|
| StdOrder | RunOrder | PtType | Blocks | concentration | Time | pH | % |
| 5 | 1 | 2 | 1 | 0.005 | 12.5 | 6 | 10.58201 |
| 8 | 2 | 2 | 1 | 0.05 | 12.5 | 8 | 21.8254 |
| 7 | 3 | 2 | 1 | 0.005 | 12.5 | 8 | 15.10141 |
| 1 | 4 | 2 | 1 | 0.005 | 1 | 7 | 7.385362 |
| 11 | 5 | 2 | 1 | 0.0275 | 1 | 8 | 14.77072 |
| 12 | 6 | 2 | 1 | 0.0275 | 24 | 8 | 22.37654 |
| 13 | 7 | 0 | 1 | 0.0275 | 12.5 | 7 | 10.25132 |
| 9 | 8 | 2 | 1 | 0.0275 | 1 | 6 | 8.928571 |
| 4 | 9 | 2 | 1 | 0.05 | 24 | 7 | 21.27425 |
| 10 | 10 | 2 | 1 | 0.0275 | 24 | 6 | 19.06966 |
| 3 | 11 | 2 | 1 | 0.005 | 24 | 7 | 14.10935 |
| 2 | 12 | 2 | 1 | 0.05 | 1 | 7 | 9.14903 |
| 15 | 13 | 0 | 1 | 0.0275 | 12.5 | 7 | 12.12522 |
| 6 | 14 | 2 | 1 | 0.05 | 12.5 | 6 | 16.86508 |
| 14 | 15 | 0 | 1 | 0.0275 | 12.5 | 7 | 15.21164 |

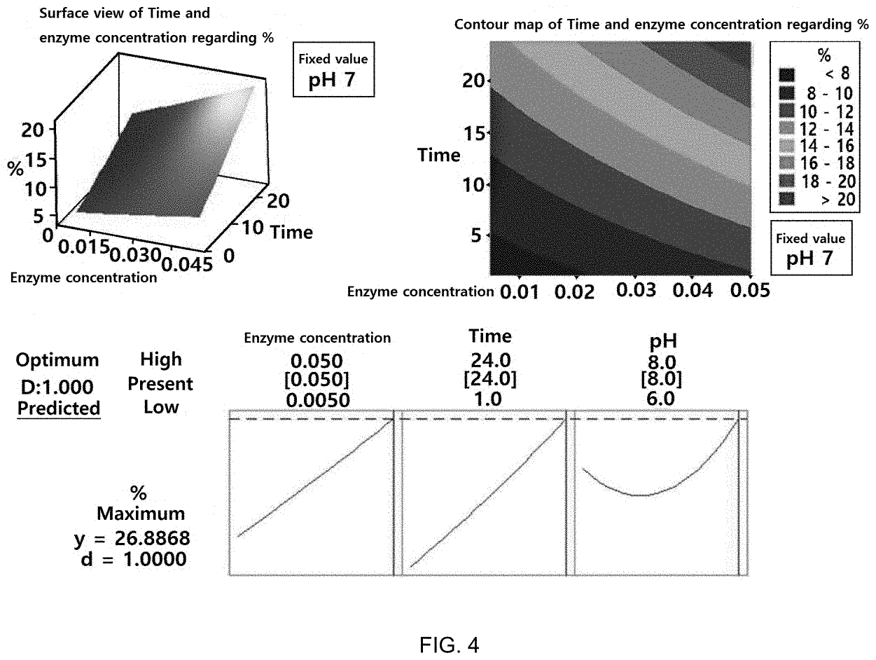

Test results are shown in Table 4 and FIG. 4. It could be seen based on these test results that an optimal yield of 26.8868% could be obtained under the conditions of an enzyme concentration of 0.05% (w/w), a treatment time of 24 hours and pH 8.0.

### Example 2: Measurement of Procollagen Synthesis Activity Depending on Type and Treatment Time of Combined Enzymes

### 1. Test Object

In the present example, procollagen synthesis activity depending on type and treatment time of combined enzymes was measured.

### 2. Procollagen Synthesis Activity Measurement Method

0.5 g of DOPC (1,2-dioleoyl-sn-glycero-3-phosphocholine), 50 mg of cholesterol and 50 μL of MPB-PE(1,2-dioleoyl-sn-glycero-3-phosphoethanolamine-N-[4-(p-maleimidophenyl)-butyramide]) were thoroughly dissolved in 10 mL of chloroform and the chloroform was then removed using a concentrator.

Then, 50 mL of PBS buffer was added to the residue and liposomes were produced by sonication. Low-molecular weight peptide (hydrolysate obtained by treating horse placenta with hydratase) was dissolved in the produced liposome, the resulting solution was stirred for 12 hours, the liposome was subjected to sonication for about 10 minutes and cells were then treated with the liposome. The amount of liposome treated was not higher than 10% of a culture medium, liposome was produced at a concentration of 50 mg/mL, and the liposome was diluted with the medium at a concentration of 1 mg/mL.

Meanwhile, WI-38 cells were cultured in minimal essential medium (MEM) 89%, supplemented with fetal bovine serum (FBS) 10% and penicillin-streptomycin (PEST) 1% in a 37° C., 5% CO₂ incubator. When the cells were cultured to 80% or more, the number of cells was sufficiently secured by subculture using trypsin-EDTA. The cells were seeded at 5×10⁵ cell/mL to a 6-well plate and were cultured until confluence reached 100% while the medium was replaced with a fresh one every two days.

The medium was replaced with FBS-free medium 12 hours before treatment of the raw material (low-molecular weight peptide-containing liposome), the medium was replaced with FBS-free medium after 12 hours and the medium was treated with the raw material. 500 μL of PBS was added to the cells washed with PBS, the cells were scraped with a scraper and centrifuged in an e-tube at 4° C. and 5,000 rpm, and the PBS was removed to produce a cell pellet.

1 mL of lysis buffer was added to the cell pellet, the pellet was moved up/down 10-20 times using a pipette, and the pellet was centrifuged at 4° C. and at 10,000 rpm to remove the cell residue. Protein was assayed by a Bradford protein assay method, 100 μL of an antibody-POD conjugate solution was mixed with 20 μL of a cell extract on a microplate equipped with a kit, and the reaction was conducted at 37° C. for 3 hours (shanking was not allowed).

After 3 hours, the reaction solution was washed with 400 μL of PBS four times, 100 μL of a substrate solution equipped with the kit was added thereto and reaction was conducted at room temperature for 15 minutes. After 15 minutes, 100 μL of sulfuric acid was added to cease the reaction, and absorbance at 450 nm of the reaction solution was measured and then compared with that of the control group, to calculate the amount of procollagen produced.

Procollagen synthesis activity=(absorbance of raw material)/(absorbance of control group)×100 [Equation 2]

### 3. Test Results

### (1) Measurement of Procollagen Synthesis Activity Depending on Type of Combined Enzymes.

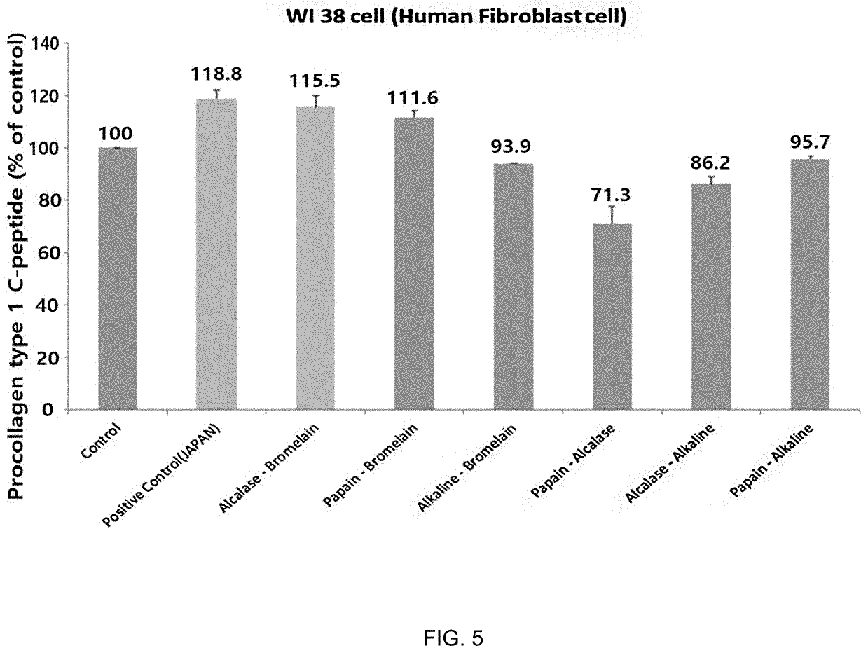

The horse placenta was treated with a combination of a variety of kinds of enzymes to obtain hydrolysates. Cells were treated with the hydrolysates and in vivo procollagen synthesis activity was confirmed. Test results confirmed that the positive control (JAPAN) had an increased procollagen synthesis activity of 118.8% as compared to the control group and a test group enzymatic-treated with Alcalase-Bromelain according to the present invention had an increased procollagen synthesis activity of 115.5% (FIG. 5). FIG. 5 shows measurement results of procollagen synthesis activity depending on type of combined enzymes.

### (2) Procollagen Synthesis Activity of Alcalase-Bromelain Combined Enzyme Over Treatment Time

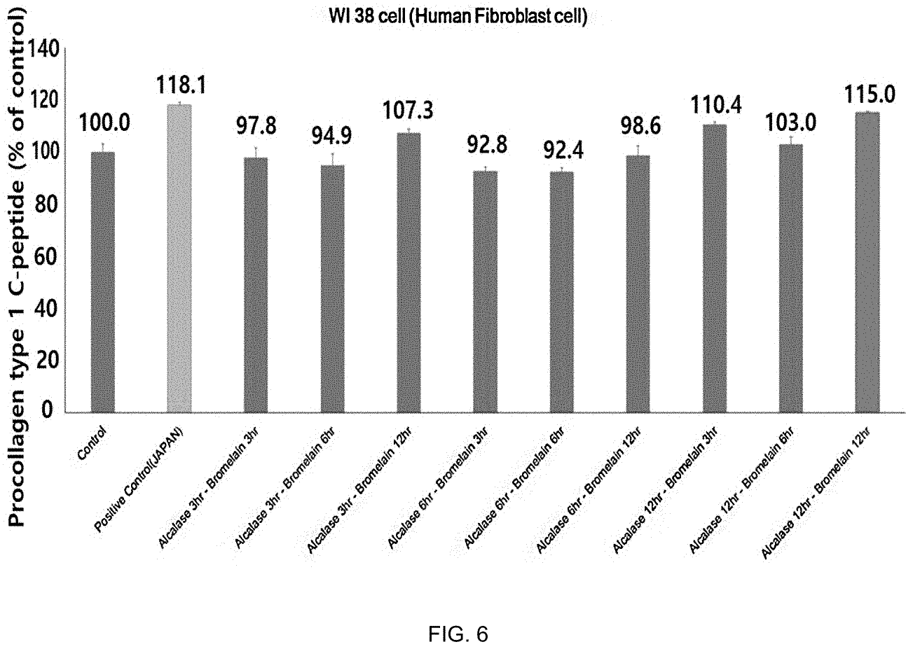

The optimal treatment time with Alcalase-Bromelain combined enzyme selected from the test was determined. As a result of determination of procollagen synthesis activity of the Alcalase-Bromelain combined enzyme, the test group treated with Alcalase for 12 hours and Bromelain for 12 hours showed the highest procollagen synthesis activity (115.0%) (FIG. 6). FIG. 6 shows measurement results of procollagen synthesis activity depending on treatment time of the Alcalase-Bromelain combined enzyme.

### Example 3: Measurement of Procollagen Synthesis Activity Upon Treatment of Exo-Type Protease

### 1. Test Object

In the present test, whether or not, in addition to treatment with the endo-type protease, treatment with exo-type protease could facilitate procollagen synthesis activity was checked.

### 2. Test Method

The test method was basically conducted in the same manner as in Example 2, except that a sample was prepared by each treating Alcalase for 12 hours and Bromelain for 12 hours as a combination of Alcalase-Bromelain enzymes. The test group was obtained by further treating this sample with an exo-type enzyme called “Flavourzyme”.

### 3. Test Results

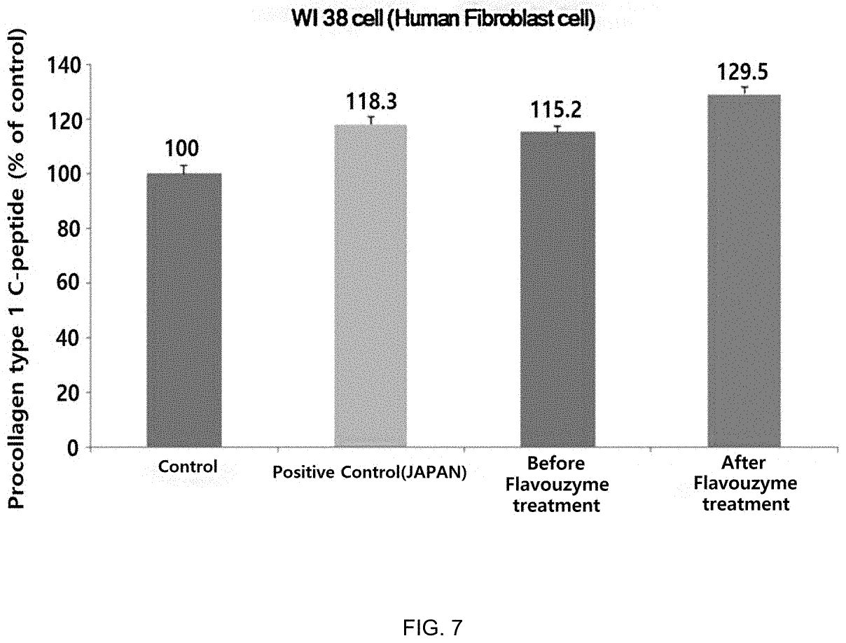

As a result of confirmation of procollagen synthesis activity involved in treatment of Flavourzyme as an exo-type protease, higher procollagen synthesis activity was observed in a Flavourzyme treatment group (129.5%), as compared to a Flavourzyme non-treatment group (115.2%) (FIG. 7). FIG. 7 shows measurement results of procollagen synthesis activity after treatment of Flavourzyme as an exo-type protease.

### Example 4: Measurement of Procollagen Synthesis Activity after Pretreatment

### 1. Test Object

In the present test, whether or not pretreatment (KOH treatment) prior to the treatment with the endo-type combined enzymes and the enzymatic treatment with an exo-type protease as described above could facilitate procollagen synthesis activity was checked.

### 2. Test Method

The test method was basically conducted in the same manner as in Example 2, except that a sample was prepared by each treating with Alcalase for 12 hours and Bromelain for 12 hours as a combination of Alcalase-Bromelain enzymes, and then treating Flavourzyme as an exo-type enzyme. The test group was a sample obtained by pre-treating with 0.1M, 0.5M and 1M of KOH before treatment of an endo-type combined enzyme and an exo-type Flavourzyme enzyme.

### 3. Test Results

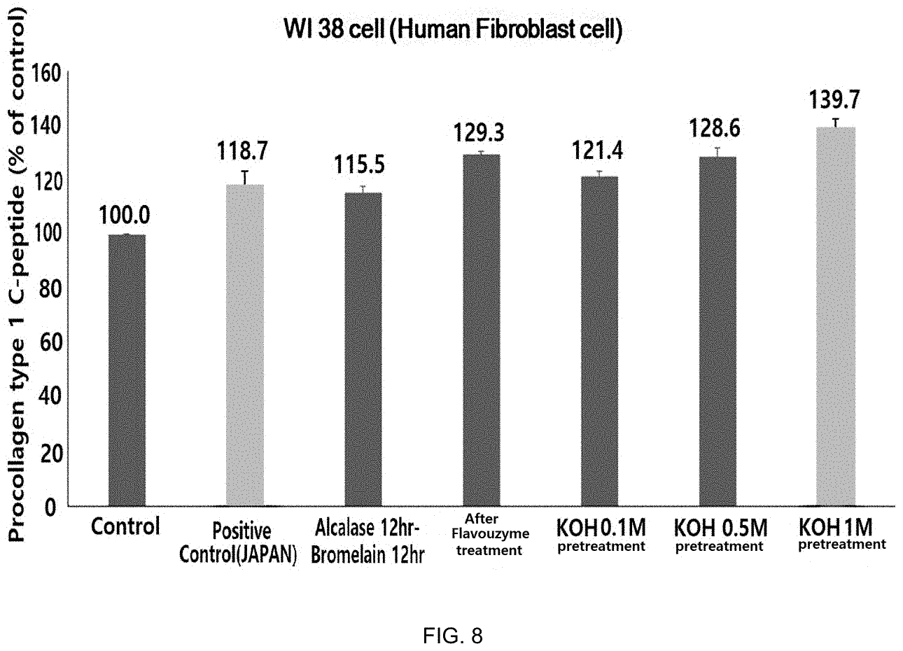

As a result of confirmation of procollagen synthesis activity after treatment with KOH at different concentrations before enzymatic treatment, procollagen synthesis activity upon 1M KOH pretreatment was found to be 139.7% (FIG. 8). FIG. 8 shows measurement results of procollagen synthesis activity after treatment with KOH before enzymatic treatment.

### Example 5: Measurement of Molecular Weight and Procollagen Synthesis Activity of Placenta Hydrolysate According to the Present Invention

### 1. Comparison and Analysis of Molecular Weight

The molecular weight of the placenta hydrolysate prepared in Example 4 according to the present invention was analyzed by MALDI-TOF.

The mass of the placenta-derived low-molecular weight peptide was analyzed using matrix-associated laser desorption ionization-time of flight mass spectrometry (MALDI-TOF, UltrafleXtreme, Bruker) in the National Center for Inter-University Research Facilities in Sungkyunkwan University. The low-molecular weight peptide was dissolved at a concentration of 5 mg/mL (5,000 ppm) in distilled water, HCCA (a-cyano-4-hydroxycinnamic acid) as a matrix was dissolved at 20 mg/mL (20,000 ppm) in a solution consisting of 0.1% acetonitrile (ACN) and trifluoroacetic acid (TFA) in a ratio of 3:7. The sample and the matrix were mixed in a ratio of 1:10, the mixture was placed on a plate for measurement and dried at room temperature, and the molecular weight was measured in a device. Measurement conditions are shown in the following Table 5.

TABLE 5

| | Reflector Mode | | Liner Mode | |
|---|---|---|---|---|
| Ion source voltage 1 | 25 | kV | 25 | kV |
| Ion source voltage 2 | 22.8 | kV | 23.2 | kV |
| Reflector voltage 1 | 26.45 | kV | 0 | kV |
| Reflector voltage 2 | 13.4 | kV | 0 | kV |
| Reflector detector | 2.484 | kV | 2.484 | kV |
| Liner detector | — | | 2.944 | kV |
| Laser beam attenuation | 30 | | 30 | |
| Laser beam focus | 35 | | 35 | |
| Laser repetition rate | 1000 | Hz | 1000 | Hz |

| | | |
|---|---|---|
| Test mode | Positive | Positive |

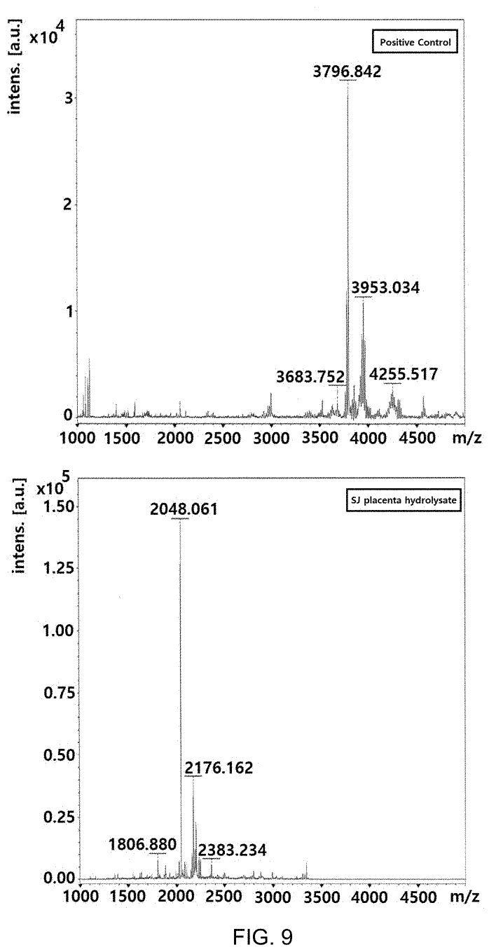

Test results confirmed that the placenta hydrolysate prepared in Example 4 according to the present invention was degraded to a lower level (2,000 to 2,500 Da) than the positive control (JAPAN) (3,500 to 4,000 Da) (FIG. 9). FIG. shows measurement results of molecular weights of the positive control (JAPAN) and placenta hydrolysate prepared in Example 4 according to the present invention.

### 2. Comparison and Analysis of Procollagen Synthesis Activity

After cells were treated with the positive control (JAPAN) and the placenta hydrolysate prepared in Example 4 according to the present invention, procollagen synthesis activity was checked. The test was conducted in the same manner as in Example 2.

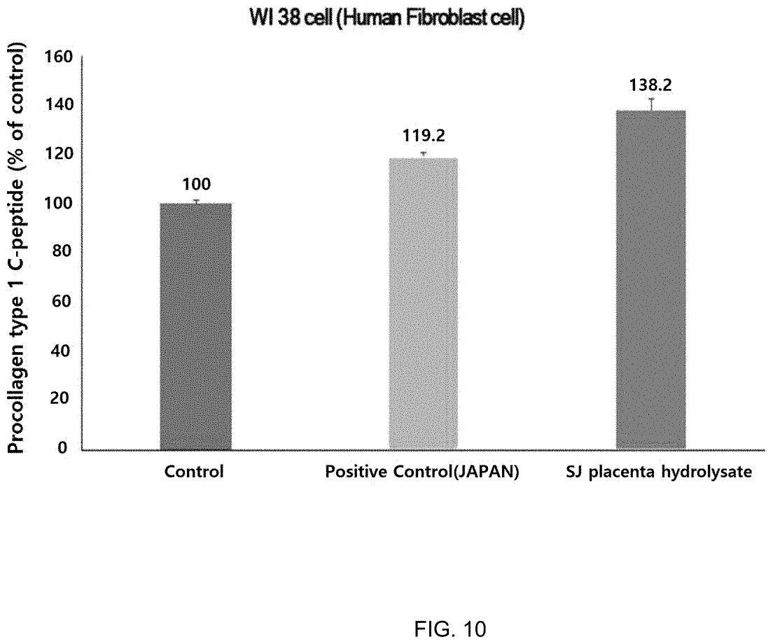

Test results confirmed that, upon treatment with the placenta hydrolysate according to the present invention, procollagen synthesis activity was found to be 138.2% (FIG. 10). FIG. 10 shows measurement results of procollagen synthesis activity of positive control (JAPAN) and the placenta hydrolysate prepared in Example 4 according to the present invention.

1. A method for preparing a horse placenta hydrolysate comprising treating a horse placenta with Alcalase, Bromelain and Flavourzyme.

2. The method according to claim 1, wherein the horse placenta is treated with at least one of Alcalase or Bromelain for 12 to 24 hours.

3. The method according to claim 1, wherein the horse placenta is treated with Flavourzyme after simultaneously treating with the Alcalase and the Bromelain.

4. The method according to claim 1, further comprising:
pretreatment of adding an alkali before the enzymatic treatment.

5. The method according to claim 4, wherein the alkali is KOH.

6. The method according to claim 1, wherein the horse placenta hydrolysate has a molecular weight of 2,000 to 2,500 Da.
